(19) United States
(12) Patent Application Publication
(10) Pub. No.: US 2025/0185971 A1
(43) Pub. Date: Jun. 12, 2025

(54) BRAIDED MULTI-ELECTRODE EMG NEEDLES FOR ADVANCED ELECTRODIAGNOSTICS

(71) Applicant: Drexel University, Philadelphia, PA (US)

(72) Inventors: Simon F. Giszter; Tae Gyo Kim; Benjamin Binder-Markey

(21) Appl. No.: 18/686,157

(22) PCT Filed: Aug. 25, 2022
(86) PCT No.: PCT/US22/41465

§ 371 Date: Feb. 23, 2024

Related U.S. Application Data
(60) Provisional application No. 63/236,955, filed on Aug. 25, 2021.

(51) Int. Cl. A61B 5/296; A61B 5/389

(52) U.S. Cl. CPC A61B 5/296; A61B 5/389; A61B 2562/125

(57) ABSTRACT

A braided needle is provided herein. The braided needle includes base needle configured for electromyography. The braided needle also includes a plurality of wires braided around an exterior surface of the base needle and along a length of the base needle, the plurality of wires being configured to transmit multiple electrical signals in connection with a biological material, the plurality of wires being further configured for electromyography.

### CROSS-REFERENCE TO RELATED APPLICATIONS

The present application claims priority under 35 U.S.C. § 119(e) to U.S. Provisional Patent Application No. 63/236,955, filed Aug. 25, 2021, which is incorporated herein by reference in its entirety.

### STATEMENT REGARDING FEDERALLY SPONSORED RESEARCH OR DEVELOPMENT

This invention was made with government support under grant numbers NINDS RO1NS072651 and NIBIB U01 EB021921 awarded by the National Institute of Health (NIH) and grant number IIS 1515140 awarded by the National Science Foundation (NSF). The government has certain rights in the invention.

### BACKGROUND

Critical diagnostic data for neural and neuromuscular disorders are collected from intramuscular electromyography (EMG). Currently, electrodiagnostic EMG is as much an art as science requiring extremely skilled individuals for the reliable acquisition and interpretation of results, which can vary from tester to tester.

It would be desirable to provide a system that simplifies the current procedures to allow consistent and rapid acquisition of EMG data.

### SUMMARY

One aspect of the invention provides a braided needle. The braided needle includes a base needle configured for electromyography. The braided needle also includes a plurality of wires braided around an exterior surface of the base needle and along a length of the base needle. The plurality of wires can be configured to transmit multiple electrical signals in connection with a biological material. The plurality of wires can be configured for electromyography

Another aspect of the invention provides a method of manufacturing a braided needle. In certain embodiments, the method includes the steps of: (a) braiding the plurality of wires onto the base needle; (b) mechanically setting the plurality of wires in a position relative to the base needle; (c) adding a volume of insulation or coating on top of the plurality of wires and the base needle; (d) cutting the plurality of wires in order to terminate the plurality of wires at a distal end of the base needle; (e) adding another volume of the insulation or the coating on top of at least a portion of the plurality of wires and the base needle; and (f) disposing a plurality of interaction sites on the plurality of wires via an ablation process.

Another aspect of the invention provides a method of using a braided needle. In certain embodiments, the method includes the steps of: (i) providing the braided needle; (ii) contacting the biological material with at least one of the plurality of wires; and (iii) transmitting the electrical signals in connection with the biological material. In certain embodiments, the method further includes the step of: conducting a multichannel analysis via the plurality of interaction sites in connection with different spatial relations to the biological material.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 illustrates a side view of a braided needle, in accordance with an exemplary embodiment of the present disclosure.

FIG. 2A illustrates a side view of a braided needle and interaction sites disposed thereon, in accordance with an exemplary embodiment of the present disclosure.

FIGS. 2B-2G illustrate various portions of interaction sites disposed on the braided needle of FIG. 2A, in accordance with an exemplary embodiment of the present disclosure.

FIG. 3 illustrates a perspective view a braided needle, in accordance with an exemplary embodiment of the present disclosure.

FIGS. 4A-4B illustrate a block diagram of a braided needle used in connection with a muscle, in accordance with an exemplary embodiment of the present disclosure.

FIG. 5 illustrates a perspective side view of a braided needle used in connection with porcine muscle, in accordance with an exemplary embodiment of the present disclosure.

FIG. 6 illustrates a flow diagram of a method of manufacturing a braided needle in accordance with an exemplary embodiment of the present disclosure.

FIG. 7 illustrates a flow diagram of a method of using the braided needle in accordance with an exemplary embodiment of the present disclosure.

FIG. 8 illustrates an example of simultaneous measurements taken in connection with a braided needle system for multichannel analysis.

FIGS. 9A-9D illustrate a comparison between three (3) single site recording EMG needles and braided needles of the present disclosure.

FIGS. 10A-10B illustrate a plurality of wires and base needle prior to and after a cutting operation.

### DETAILED DESCRIPTION OF THE INVENTION

The present disclosure describes a braided needle and/or a multi-electrode probe used for neural recording and stimulation. Braided needles can use highly flexible ultrafine wires in a tubular microbraid. Braids add capabilities in electrophysiology, such as allowing chronic recording & stimulation in brains and especially in spinal cords in freely moving animals. Multisite recording benefits can allow clinicians to obtain several examples of single motor units simultaneously by a single insertion (in contrast to other EMG needles which require multiple insertions with a single recording site, often at the end of the probe), improving signal reliability and reducing pain. A novel EMG needle having microwires braided onto a conventional EMG needle offering multi-recording sites are described in the present disclosure. A manufacturing process producing such a needle is described herein. To braid a plurality of wires onto a base needle, a braiding machine may be utilized. The combination of the braid of a plurality of needles and a base needle can undergo a heating process to adhere the plurality of wires (e.g., by partial melt, fusion, etc.) the plurality of wires and base needle for structural integrity of the assembly as needed for certain applications (e.g., smooth insertions). For fine wire cutting at microscale, a mechanism for a 360° even cut can be implemented. Interaction sites (e.g., recording sites) are made on each wire by ablation (e.g., laser ablation).

Electromyography (EMG) needles having multiple electrodes for multichannel EMG motor unit potentials (MUPs) and multiple isolated single motor unit (SMU) recordings are described herein. The needle is created with braided wires (e.g., microwires). Braids add capabilities in electrophysiology: specifically, allowing chronic recording & stimulation in brains and especially in spinal cords in freely moving animals. The microwires are braided onto an EMG needle and fixed in place in precise geometries to significantly improve signal reliability through multichannel acquisition, and thereby to gain more MUP and SMU signals per location and thereby reduce manipulation needed, and patients' pain during the electrodiagnostic procedure. Such collected data sets can be suited to machine learning and automated multichannel analysis.

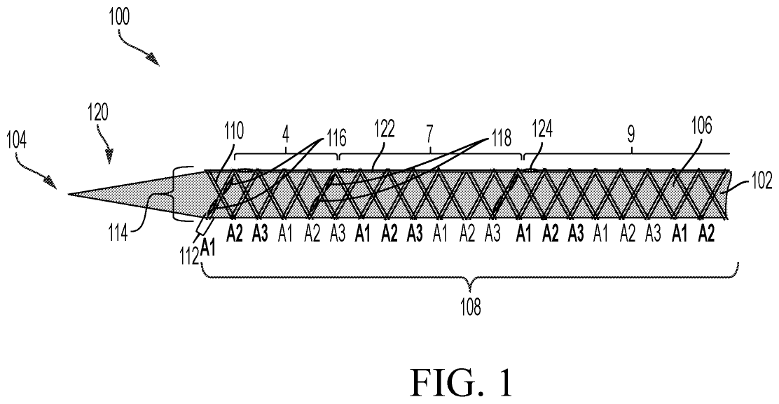

The invention is best described in connection with the various drawings, where like elements have like reference numerals across the various drawings. Referring now to FIG. 1, a braided needle 100 (e.g., an electromyography (EMG) needle having multiple electrodes) is illustrated. Braided needle 100 includes a base needle 102 configured for electromyography. Base needle 102 includes an elongated portion 108 extending along the length of base needle 102. Base needle 102 includes a tip portion 104 at a distal end 120 of base needle 102.

Braided needle 100 also includes a plurality of wires 110. The plurality of wires 110 are arranged in a braided configuration around an exterior surface 106 of base needle 102, forming braid 114. The plurality of wires 110 can be grouped into grouping 112 (e.g., two wires, three wires, four wires, five wires, six wires, seven wires, eight wires, etc.). The plurality of wires 110 (and/or the groupings 112) are arranged in a helical pattern around base needle 102. The plurality of wires 110 (and/or the groupings 112) are illustrated interleaved with (or wrapped around) a lay-in structure 122 (e.g., a lay-in wire) running along the length of base needle 102. The plurality of wires 110 terminate at distal end 120 of the base needle 102. The plurality of wires 110 are illustrated including a plurality of interaction sites (e.g., recording sites, communication sites, transmission sites, etc.). Interaction sites 116 (and/or interaction sites 118) are disposed on at least some of the plurality of wires 110. Interaction sites can be disposed on wires 110 at certain locations using an ablation process. Interaction sites 124 (e.g., reference interaction sites, reference recording sites, etc.) are disposed along the along lay-in structure 122. Interaction sites 116 can be disposed on certain portions of certain wires 110 in groupings (e.g., grouping 112).

The plurality of interaction sites (e.g., interaction sites 116 of grouping 112 and interaction sites 124 of lay-in structure 122) are configured to measure an electrical signal from biological material (e.g., muscle tissue). The electrical signal of a biological material may be generated in a muscle associated with motor activity or pathological motor behavior (or resulting from the delivery of electrical current). The electrical signal of the biological material may be motor unit potentials, single motor units, single fiber potentials, electrical voltage or signals needed to estimate electrical impedance (e.g., a current-controlled or voltage-controlled sinewave function input may be used to measure signals used to derive impedance, resistance, inductance, capacitance, etc.). The plurality of interaction sites of braided needle 100 can be brought into contact with biological material at various locations. The relationship (e.g., spatial relationship) between each of interaction sites 116 and interaction sites 124 can be used to perform multichannel analyses.

The plurality of wires 110 can be made of a material suitable for use in connection with braiding and/or electromyography. In certain embodiments, the plurality of wires 110 can be made from a biocompatible or non-toxic alloy. In certain embodiments, the plurality of wires 110 electrically conductive material in a fully hermetic encapsulation. In certain embodiments, plurality of wires 110 can be made (at least in part) from nichrome, steel, platinum, platinum iridium, gold, or other suitable materials. In certain application, a coating (e.g., polyimide, a biocompatible electrical insulation material suited to braiding and ablation, etc.) may be applied to the plurality of wires 110.

Base needle 102 can be made from certain stiff and robust materials suitable for making a penetrating needle. In certain embodiments, base needle 102 can be made from certain non-toxic and biocompatible materials (e.g., with regard to the duration the needle is in contact with biological material. In certain embodiments, base needle 102 can be made (at least in part) from carbon (e.g., with suitable form and structural material composition). In certain applications, base needle 102 can be coated with a material with a low friction, adhesive material, non-toxic, and/or biocompatible material. In certain applications, base needle 102 can be coated with (at least in part) PTFE, silicone, or a similar composition.

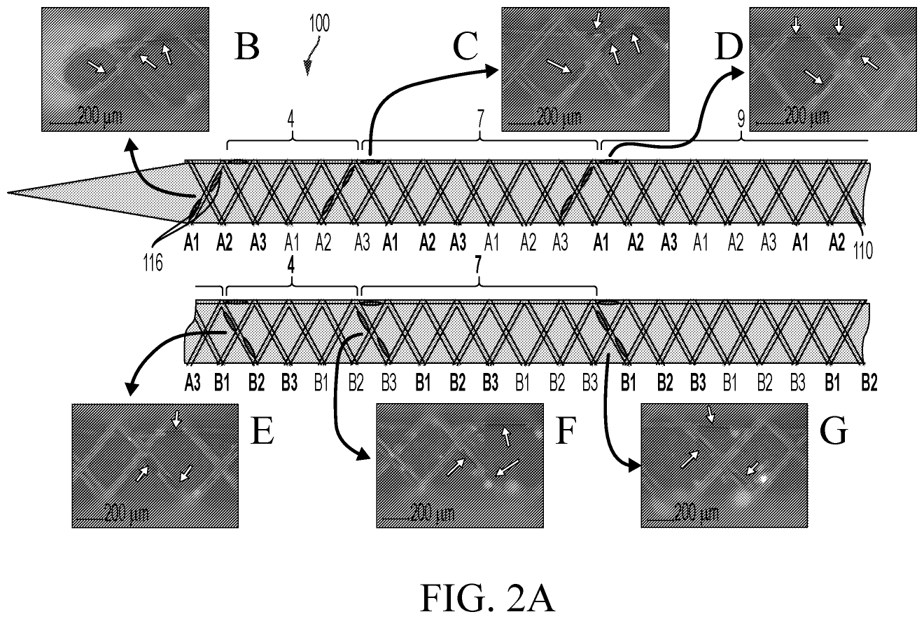

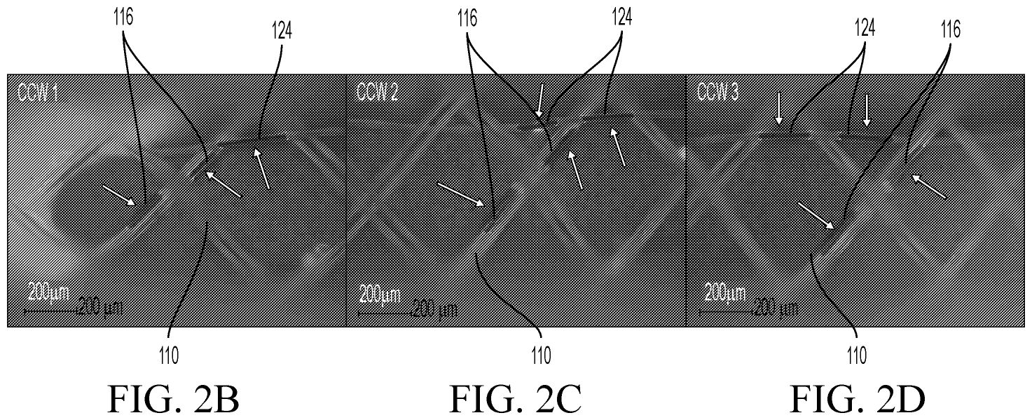

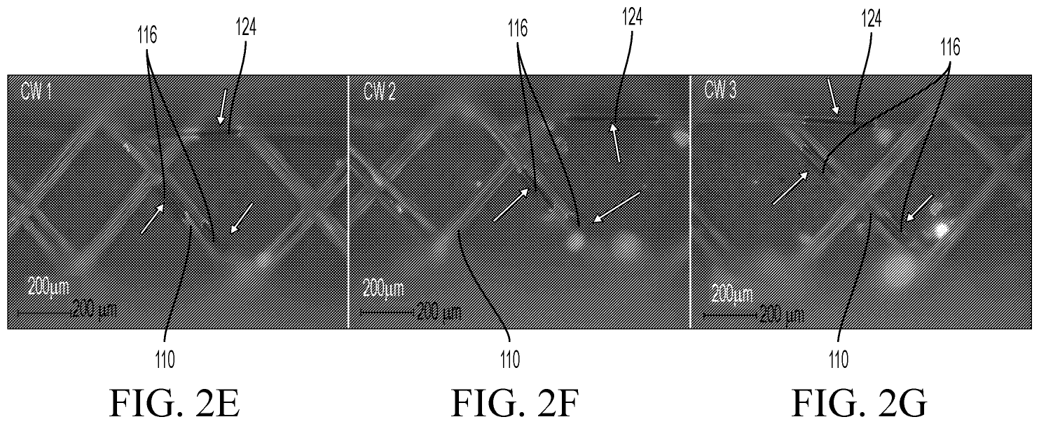

Referring now to FIG. 2A, braided needle 100, with a plurality of interaction sites 116, is illustrated. FIGS. 2B-2G more clearly illustrate various interaction sites, and their positional relationship to each other. The interaction sites may be varied in size and placement (i.e., absolute placement or relative placement) depending on the desired measurement (e.g., using multichannel analysis, signal triangulation, etc.) or desired use of the interaction sites (e.g., measuring electrical signals, transmitting electrical signals in a stimulation application, etc.)

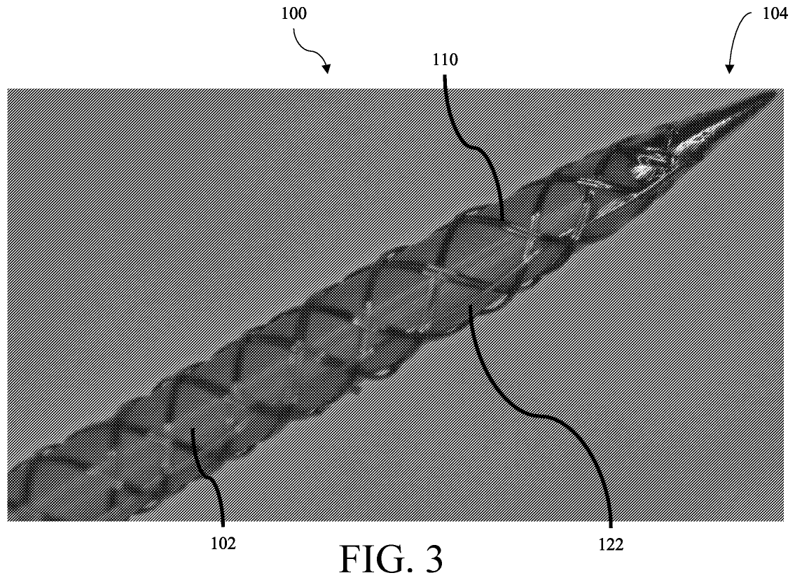

Referring now to FIG. 3, a perspective view of a braided needle 100 is illustrated.

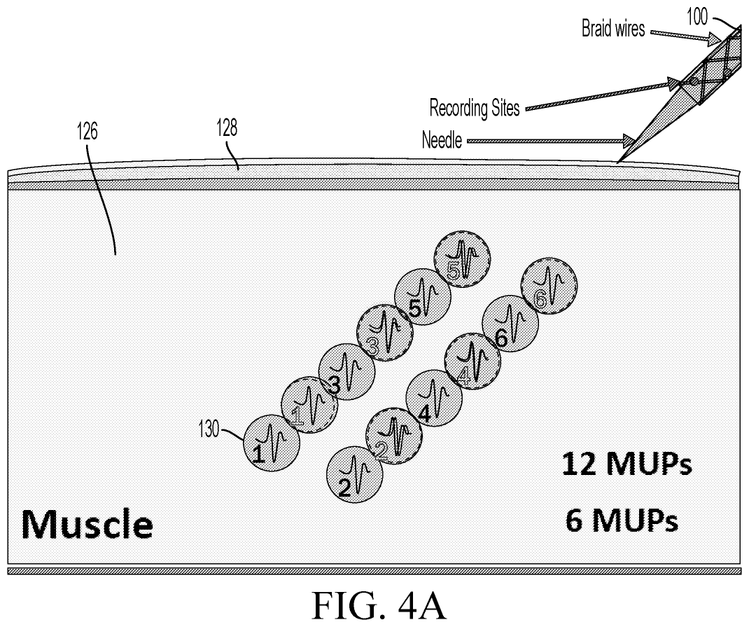

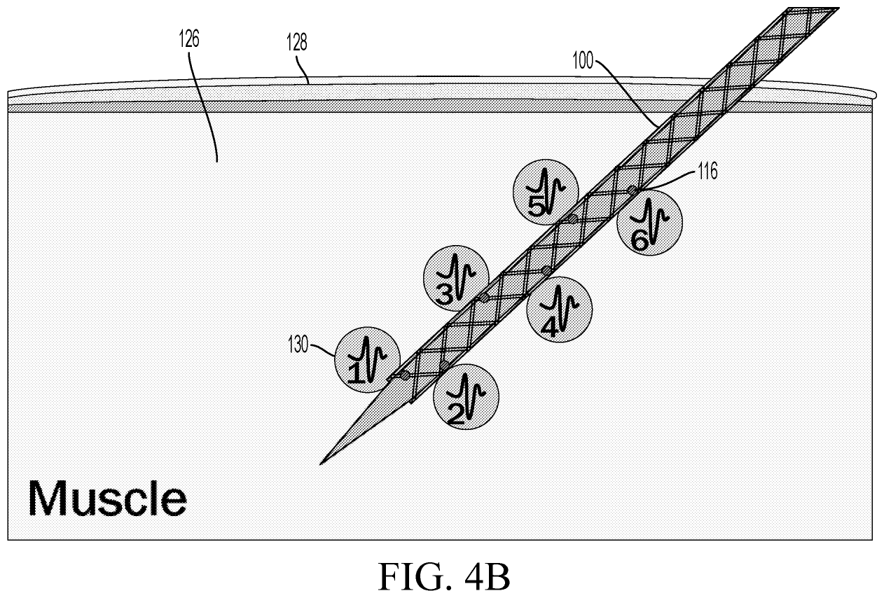

Referring now to FIGS. 4A-4B, block diagrams of braided needle 100 being used in connection with a biological material 126 (e.g., muscle) are illustrated. Braided needle 100 is positioned just outside of another biological material 128 (e.g., skin) prior to being inserted through biological material 126 (e.g., muscle). After being inserted, a plurality of interaction sites (e.g., recording sites) can make contact with biological material 126 (e.g., muscle) such that a plurality of electrical signal sources 130 (e.g., MUPs) of biological material 126 can be measured. Such a use of braided needle 100 offers the advantage of being able to take multiple measurements (e.g., of electrical signal sources 130) from multiple locations (e.g., serially or simultaneously) without moving the tip of a needle around to take multiple measurements, which would cause some negative consequences (e.g., pain, discomfort, greater risk of injury or damage to the biological material, etc.). In FIG. 4B, a multichannel analysis can be conducted via the plurality of interaction sites 116 in connection with different spatial relations to the biological material (e.g., the different spatial relationships of electrical signal sources 130).

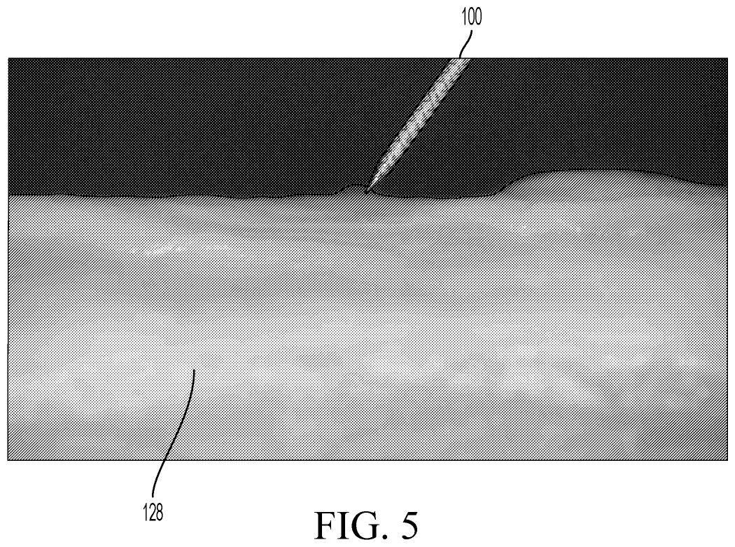

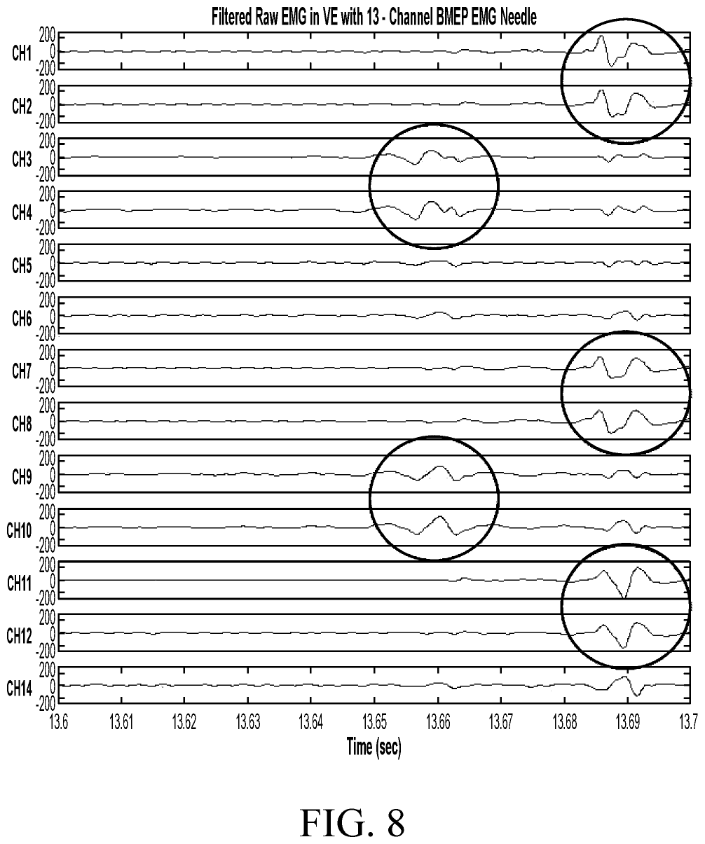

A depiction of braided needle 100 being used in connection with a porcine muscle sample biological material is illustrated in FIG. 5. An example of measurements taken from a multichannel analysis from living muscle is illustrated in FIG. 8. In certain embodiments, the multichannel analysis or measurement is done simultaneously, as illustrated in FIG. 8.

The manufacturing processes for braided needles are also described herein. The manufacture required includes a plurality of processing steps, including: providing a base needle; braiding a plurality of insulated wires (e.g., insulated wires, ultrafine wires, insulated ultrafine wires, etc.) onto a base needle; heating the base needle and the plurality of wires to set the wires in place; adding a biocompatible target insulation (e.g., polytetrafluoroethylene (PTFE)); cut the plurality of wires (e.g., removing wire not in contact with the exterior surface of base needle; removing wire beyond the tip of the need, etc.); applying further insulation (e.g., PTFE insulation, PTFE cut region insulation, etc.); and/or laser ablation of the braided needle (e.g., laser ablation of sites on the braided needle wires; disposing interaction sites on the plurality of wires, etc.).

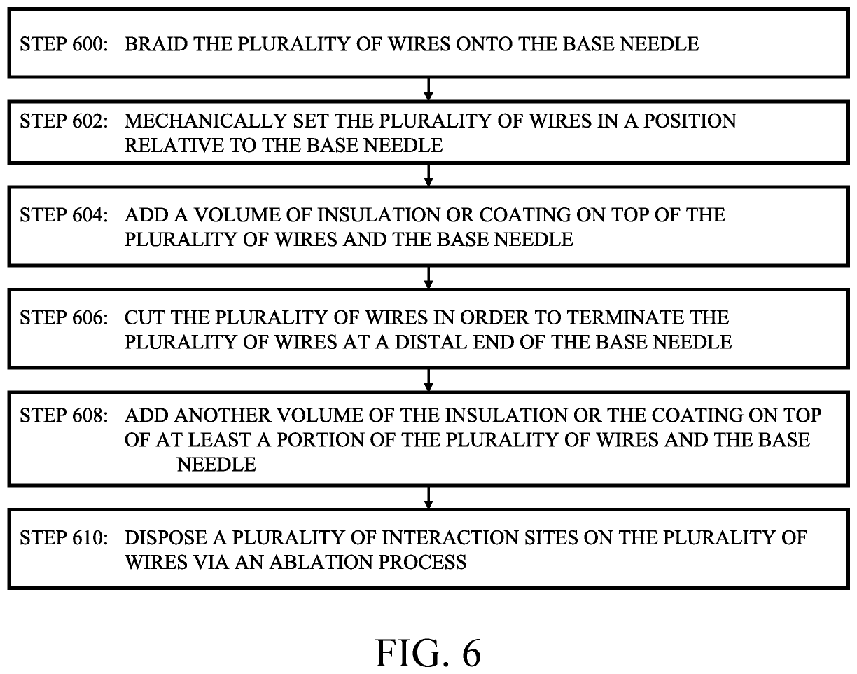

Referring now to FIG. 6, a flow diagram depicting a method of manufacturing a braided needle is illustrated, in accordance with exemplary embodiments of the present disclosure. As is understood by those skilled in the art, certain steps included in the flow diagram may be omitted; certain additional steps may be added; and the order of the steps may be altered from the order illustrated.

In step 600, the plurality of wires is braided onto the base needle. For example, the wires may be braided into a helical pattern (or another pattern) around an exterior surface of a base needle (e.g., for electromyography). In certain embodiments, microwires (e.g., wires with a diameter of 10 um or less, micro wires with a 10.2 um diameter and Nichrome composition with PTFE insulation, a wire with a polyimide or other biocompatible material; etc.) are braided onto a base needle (e.g., any of one of several EMG needle designs). In certain embodiments, the number of microwires can comprise 6 wires. In certain embodiments, the number of microwires can comprise 24 wires. In certain embodiments, the number of microwires can comprise a plurality of wires (e.g., in multiples of 6 wires). Step 600 may be implements using a braiding apparatus system. In certain embodiments, the wires have single recording sites. Since each microwire can have a single independent channel for EMG recording, the number of microwires used in a braided multi-electrode EMG needle can be the same as the number of recording channels. In other embodiments, the single wires may have multiple sites ablated on each, allowing combinatoric recording. In yet other embodiments, paired or grouped wires with single sites are used differentially (e.g., in recording; in measuring; in stimulating, and so forth).

In step 602, the plurality of wires are mechanically set in a position relative to the base needle. In certain embodiments, the base needle and the plurality of wires insulation or coating is heated to set the wires in a position with relative to the base needle, the wires including an insulation. Step 602 can include a high temperature heating process to make a material fusion between the wire insulation (e.g., microwire PTFE insulation, other biocompatible target insulation, etc.) and the insulation (e.g., PTFE insulation) on the base needle body fusing the wires (e.g., microwires) and the base needle body into an integral mechanical unit. Such an integral mechanical unit can be useful for smooth insertion into biological materials (e.g., muscles, muscle tissue, etc.). In certain other embodiments, the plurality of wires can be mechanically set with a non-thermal curing insulation or coating (e.g., silicone coating).

In step 604, a volume of insulation or coating is added on top of the plurality of wires and the base needle. In certain embodiments, a thin coating layer over the braided wires on the base needle is made with PTFE solution or another needle coating material. Such a layer/coating is configured to prevent wire flaring (e.g., in subsequent cutting processes) and/or providing additional outer surface coating and smoothness of the braided needle (e.g., braided EMG needle).

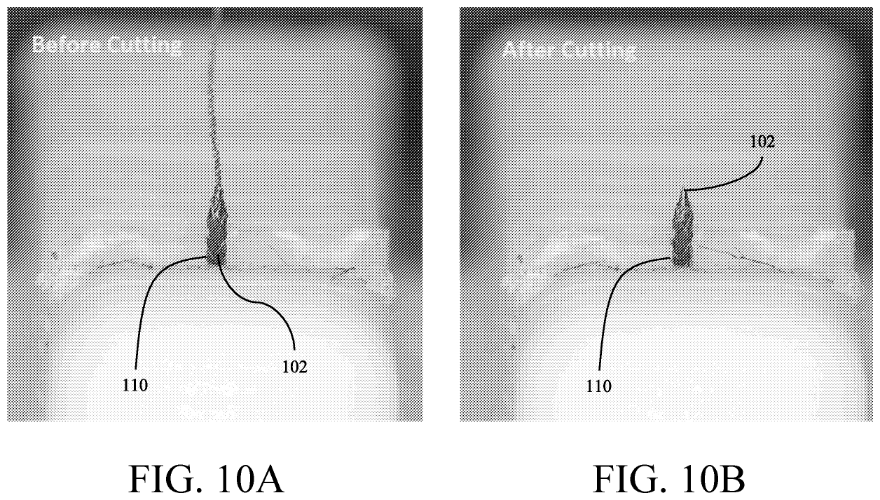

In step 606, the plurality of wires is cut in order to terminate the plurality of wires at a distal end of the base needle. In certain embodiments, step 606 cuts wires held in place (e.g., by PTFE or another coating) close to the tip of a base needle. In certain embodiments, a 360° cutting mechanism may be used for microscale precise cutting. The plurality of wires 110 and base needle 102 are illustrated prior to a cutting operation in FIG. 10A. The plurality of wires 110 and base needle 102 are illustrated after a cutting operation in FIG. 10B.

In step 608, another volume of the insulation or the coating is added on top of at least a portion of the plurality of wires and the base needle. In certain embodiments, the tips of wires (e.g., which are exposed by the cutting of step 606) are insulated with a layer or coating (e.g., PTFE, another insulator of choice, etc.). In certain embodiments, the section of wires (and/or the base needle) affected by a cutting operation (or another mechanical operation) may be coated or insulated. Such a layer/coating can be configured to provide additional outer surface coating and smoothness of the braided needle (e.g., braided EMG needle).

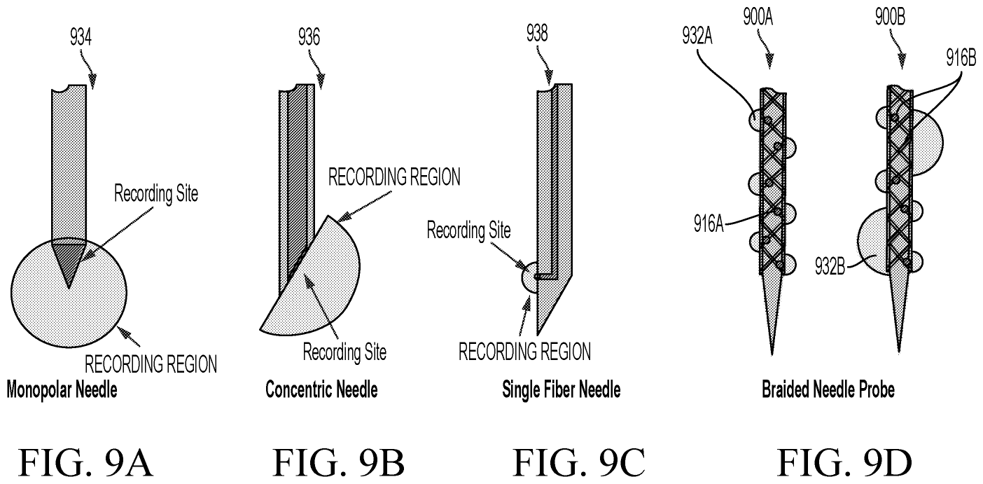

In step 610, a plurality of interaction sites are disposed on the plurality of wires via an ablation process. In this step, the interaction sites (e.g., recording sites) are exposed on individual microwires via PTFE (or other insulation) removals by ablation (e.g., laser ablation). The positions and exposed areas of individual interaction sites (e.g., recording sites) can be customized depending on applications as noted above. The area size of an exposed site determines site electrical impedance and/or the signal precision of the site. For example, the characteristics of three sites (e.g., see interaction sites 916A/916B of braided needle 900A/900B of FIG. 9D) could be equivalent to one of three other types of single site recording EMG needles (e.g., see monopolar needle 934, concentric needle 936, or single fiber needle 938 of FIGS. 9A-9C), which can produce various mixed area sizes of sites on the same braided multi-electrode EMG needle (e.g., including multichannel and/or multitype analysis).

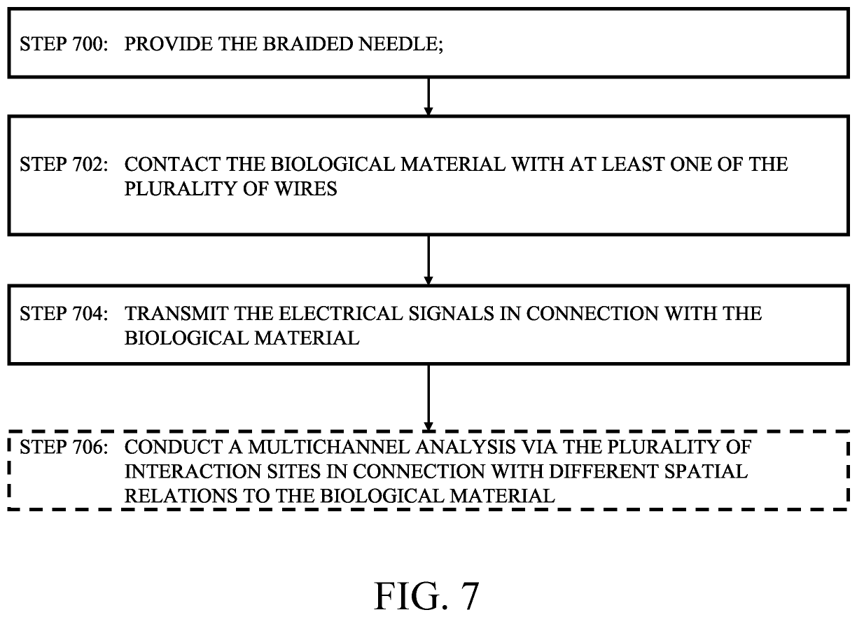

Referring now to FIG. 7, a method of using the braided needle is illustrated. In step 700, the braided needle (e.g., multichannel EMG needle) is provided. In step 702, the biological material is contacted with at least one of the plurality of wires. In step 704, the electrical signals are transmitted (e.g., measured from, transmitted to, etc.) in connection with the biological material. In step 706, multichannel analysis is conducted via the plurality of interaction sites in connection with different spatial relations to the biological material.

Instead of using conventional EMG needles, custom built tungsten or other alloy needles with PTFE (or polyimide or other insulation) could be used for the braided multi-electrode EMG needle base, to further reduce the needle diameter and ease muscle penetration and pain. Other EMG needles (e.g., monopolar, concentric, single fiber, etc.) being used in contemporary electrodiagnostics have only a single channel for recording single motor units at or around the sharp tips of needles. Thus, such EMG needles are inserted into a target muscle multiple times by changing depths and insertion angles in order to reposition the tip as needed to obtain multiple (e.g., 12-15) motor units needed for standard of care diagnosis. Such procedures decreases signal reliability across clinicians and increases pain by multiple insertions.

The braided needle (e.g., braided multi-electrode EMG needle) of the present disclosure provides multiple (e.g., more than 1, 6-24, more than 24, etc.) channels for EMG recording along the EMG needle length, depending on the number of wires braided on a needle. Referring now to FIGS. 9A-9D generally and FIG. 9D specifically, braided needle 900A and braided needle 900B are illustrated. Braided needle 900A and braided needle 900B are substantially similar to braided needle 100 illustrated throughout the various drawings, except interaction sites 916A and 916B (comparable to the elements labeled “recording site” in FIGS. 9A-9C) are illustrated to accompany recording regions 932A and 932B (comparable to the elements labeled “recording region” in FIGS. 9A-9C). Recording regions 932A and 932B are recording regions of useful signal acquisition of a biological material (e.g., a muscle). Braided needles 900A/900B provide advantages over single site monopolar needle 934, concentric needle 936, or single fiber needle 938. For example, the interaction sites 916A/916B may serve in lieu of the various recording sites of monopolar needle 934, concentric needle 936 and/or single fiber needle 938 in order to measure a signal (e.g., electrical signal) of the multiple recording regions 932A/932B (in lieu of the various recording areas of monopolar needle 934, concentric needle 936 and/or single fiber needle 938). The advantages of braided needles 900A-900B (e.g., over monopolar needle 934, concentric needle 936 and/or single fiber needle 938) include simultaneous measurements of multiple regions to reduce pain (from multiple insertions of needles), higher signal reliability, richer signal multivariate capabilities, faster overall measurement procedures, and other advantages. It should be understood that recording regions 932A/932B can also describe the general regions of the biological material which may be stimulated by the interaction sites.

Thus, a one-time deep insertion of the braided needle (e.g., braided EMG needle) can simultaneously obtain multiple (e.g., 6-24) different motor units simultaneously. By pulling the braided needle back several times, many more distinct/different motor units can be additionally obtained/measured. Such a procedure with the braided needle allows clinicians to observe and compare multiple (e.g., 2, 3, 4, 5, 6, 7, 8, 9, 10, 11, 12, etc.) motor units at once and provides the means to generate statistical measures and outputs across multiple channels, which results in much more objective EMG outputs for diagnosis in an automated and faster way. This significantly increases EMG signal reliability with consistent and more objective data and at the same time reduces pain by fewer insertions (or even a single insertion). The braided needle (e.g., multi-electrode EMG needle) has several advantages over other EMG needles, including higher signal reliability, richer signal multivariate capabilities, faster overall measurement procedures, and pain reduction.

### ENUMERATED EMBODIMENTS

The following enumerated embodiments are provided, the numbering of which is not to be construed as designating levels of importance.

Embodiment 1 provides a braided needle, comprising: a base needle configured for electromyography; and a plurality of wires braided around an exterior surface of the base needle and along a length of the base needle, the plurality of wires being configured to transmit multiple electrical signals in connection with a biological material, the plurality of wires being further configured for electromyography.

Embodiment 2 provides the braided needle of embodiment 1, wherein the plurality of wires comprises microwires.

Embodiment 3 provides the braided needle of any one of embodiments 1-2, further comprising a plurality of interaction sites disposed on at least some of the plurality of wires.

Embodiment 4 provides the braided needle of any one of embodiments 1-3, wherein the plurality of interaction sites comprises recording sites.

Embodiment 5 provides the braided needle of any one of embodiments 1-4, wherein the plurality of interaction sites are disposed on the plurality of wires using an ablation process.

Embodiment 6 provides the braided needle of any one of embodiments 1-5, wherein the plurality of interaction sites are configured to measure an electrical signal from the biological material, the electrical signal being generated in a muscle associated with motor activity or pathological motor behavior, the electrical signal of the biological material being selected from the group consisting of: motor unit potentials, single motor units, single fiber potentials, electrical voltage and electrical impedance.

Embodiment 7 provides the braided needle of any one of embodiments 1-6, wherein a fraction of the plurality of interaction sites are configured to stimulate the biological material with electrical signals provided by the braided needle.

Embodiment 8 provides the braided needle of any one of embodiments 1-7, further comprising a lay-in structure positioned along the length of the base needle, wherein each of the plurality of wires is braided in a helical pattern around the base needle.

Embodiment 9 provides the braided needle of any one of embodiments 1-8, wherein each wire of the plurality of wires is part of a grouping of wires, the grouping of wires being selected from the group consisting of: two wires; three wires; four wires; five wires; six wires; seven wires; and eight wires.

Embodiment 10 provides the braided needle of any one of embodiments 1-9, wherein the plurality of wires includes at least four wires.

Embodiment 11 provides the braided needle of any one of embodiments 1-10, wherein at least two wires of the plurality of wires terminate at a distal end of the base needle.

Embodiment 12 provides a method of manufacturing the braided needle of any one of embodiments 1-11, comprising the steps of: (a) braiding the plurality of wires onto the base needle; (b) mechanically setting the plurality of wires in a position relative to the base needle; (c) adding a volume of insulation or coating on top of the plurality of wires and the base needle; (d) cutting the plurality of wires in order to terminate the plurality of wires at a distal end of the base needle; (e) adding another volume of the insulation or the coating on top of at least a portion of the plurality of wires and the base needle; and (f) disposing a plurality of interaction sites on the plurality of wires via an ablation process.

Embodiment 13 provides the method of manufacturing of embodiment 12, wherein the ablation process is a laser ablation process.

Embodiment 14 provides the method of manufacturing of any one of embodiments 12-13, wherein the interaction sites are recording sites

Embodiment 15 provides the method of manufacturing of any one of embodiments 12-14, wherein step (b) includes heating the base needle and the plurality of wires insulation or coating to set the wires in a position relative to the base needle.

Embodiment 16 provides the method of manufacturing of any one of embodiments 12-15, wherein step (b) includes mechanically setting the plurality of wires with a non-thermal curing insulation or coating.

Embodiment 17 provides a method of using the braided needle of any one of embodiments 1-16, comprising the steps of: (i) providing the braided needle; (ii) contacting the biological material with at least one of the plurality of wires; and (iii) transmitting the electrical signals in connection with the biological material.

Embodiment 18 provides a method of using the braided needle of any one of embodiments 1-17, wherein the plurality of wires includes a plurality of interaction sites disposed on at least some of the plurality of wires configured for combinatoric analysis.

Embodiment 19 provides a method of using the braided needle of any one of embodiments 1-18, wherein one interaction site is disposed on each wire.

Embodiment 20 provides a method of using the braided needle of any one of embodiments 1-19, wherein multiple interaction sites are disposed on each wire.

Embodiment 21 provides a method of using the braided needle of any one of embodiments 1-20, wherein step (ii) includes contacting the biological material with at least one of the plurality of interaction sites.

Embodiment 22 provides a method of using the braided needle of any one of embodiments 1-21, further comprising the step of: (iv) conducting multichannel analysis via the plurality of interaction sites in connection with different spatial relations to the biological material.

Embodiment 23 provides a method of using the braided needle of any one of embodiments 1-22, wherein the electrical signals are measured from the biological material.

Embodiment 24 provides a method of using the braided needle of any one of embodiments 1-23, wherein the electrical signals are generated in a muscle associated with motor activity or pathological motor behavior, wherein at least one of the electrical signals is selected from the group consisting of: motor unit potentials, single motor units, single fiber potentials, electrical voltage and electrical impedance.

Embodiment 25 provides a method of using the braided needle of any one of embodiments 1-24, wherein the electrical signals are transmitted to the biological material by the delivery of electrical current at selected interaction sites.

Embodiment 26 provides a method of using the braided needle of any one of embodiments 1-25, wherein the electrical signals are transmitted to and from the biological material by the delivery of electrical current at selected interaction sites.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A braided needle, comprising:
a base needle configured for electromyography; and
a plurality of wires braided around an exterior surface of the base needle and along a length of the base needle, the plurality of wires being configured to transmit multiple electrical signals in connection with a biological material, the plurality of wires being further configured for electromyography.

2. The braided needle of claim 1, wherein the plurality of wires comprises microwires.

3. The braided needle of claim 1, further comprising a plurality of interaction sites disposed on at least some of the plurality of wires.

4. The braided needle of claim 3, wherein the plurality of interaction sites comprises recording sites.

5. (canceled)

6. The braided needle of claim 3, wherein the plurality of interaction sites are configured to measure an electrical signal from the biological material, the electrical signal being generated in a muscle associated with motor activity or pathological motor behavior, the electrical signal of the biological material being selected from the group consisting of: motor unit potentials, single motor units, single fiber potentials, electrical voltage and electrical impedance.

7. The braided needle of claim 3, wherein a fraction of the plurality of interaction sites are configured to stimulate the biological material with electrical signals provided by the braided needle.

8. The braided needle of claim 1, further comprising a lay-in structure positioned along the length of the base needle, wherein each of the plurality of wires is braided in a helical pattern around the base needle.

9. The braided needle of claim 1, wherein each wire of the plurality of wires is part of a grouping of wires, the grouping of wires being selected from the group consisting of:
two wires; three wires; four wires; five wires; six wires; seven wires; and eight wires.

10. The braided needle of claim 1, wherein the plurality of wires includes at least four wires.

11. The braided needle of claim 1, wherein at least two wires of the plurality of wires terminate at a distal end of the base needle.

12. A method of manufacturing the braided needle of claim 1, comprising the steps of:
(a) braiding the plurality of wires onto the base needle;
(b) mechanically setting the plurality of wires in a position relative to the base needle;
(c) adding a volume of insulation or coating on top of the plurality of wires and the base needle;
(d) cutting the plurality of wires in order to terminate the plurality of wires at a distal end of the base needle;
(e) adding another volume of the insulation or the coating on top of at least a portion of the plurality of wires and the base needle; and
(f) disposing a plurality of interaction sites on the plurality of wires via an ablation process.

13. The method of claim 12, wherein the ablation process is a laser ablation process.

14. (canceled)

15. The method of claim 12, wherein step (b) includes heating the base needle and the plurality of wires insulation or coating to set the wires in a position relative to the base needle.

16. The method of claim 12, wherein step (b) includes mechanically setting the plurality of wires with a non-thermal curing insulation or coating.

17. A method of using the braided needle of claim 1, comprising the steps of:
(i) providing the braided needle;
(ii) contacting the biological material with at least one of the plurality of wires; and
(iii) transmitting the electrical signals in connection with the biological material.

18. The method of claim 17, wherein the plurality of wires includes a plurality of interaction sites disposed on at least some of the plurality of wires configured for combinatoric analysis.

19. (canceled)

20. (canceled)

21. The method of claim 18, wherein:
step (ii) includes contacting the biological material with at least one of the plurality of interaction sites; and
the electrical signals are transmitted to and from the biological material by the delivery of electrical current at selected interaction sites.

22. The method of claim 18, further comprising the step of:
iv) conducting a multichannel analysis via the plurality of interaction sites in connection with different spatial relations to the biological material.

23. The method of claim 18, wherein the electrical signals are measured from the biological material.

24. The method of claim 23, wherein the electrical signals are generated in a muscle associated with motor activity or pathological motor behavior, wherein at least one of the electrical signals is selected from the group consisting of: motor unit potentials, single motor units, single fiber potentials, electrical voltage and electrical impedance.

25. (canceled)

26. (canceled)
